(19) United States
(12) Patent Application Publication
(10) Pub. No.: US 2024/0245884 A1
(43) Pub. Date: Jul. 25, 2024

(54) FIBROUS ARAMID REINFORCED CATHETER

(71) Applicant: Merit Medical Systems, Inc., South Jordan, UT (US)

(72) Inventors: Wayne Mower, Bountiful, UT (US); John Hall, Bountiful, UT (US); Christopher Cindrich, Highland, UT (US); Michael Adams, Bluffdale, UT (US); Christian Lentz, Sandy, UT (US); Muluken Terefe, Richmond, TX (US); Adam Wilhite, Herriman, UT (US)

(21) Appl. No.: 18/421,747

(22) Filed: Jan. 24, 2024

Related U.S. Application Data
(60) Provisional application No. 63/481,463, filed on Jan. 25, 2023.

(51) Int. Cl. A61M 25/00; A61L 29/04

(52) U.S. Cl. CPC A61M 25/005; A61L 29/04; A61M 2025/0004; A61M 2205/02

(57) ABSTRACT

A vascular prosthesis deployment device and related methods are disclosed. In some embodiments the deployment device may include a delivery catheter assembly. The delivery catheter assembly may include an outer sheath, an intermediate sheath, and an inner sheath. One or more of the outer sheath, the intermediate sheath, and the inner sheath may be reinforced with a braided structure to prevent elongation and ovalization of the sheaths. The braided structure may include one or more yarn members imbedded in a material and formed from a plurality of twisted singles. The twisted singles may be formed from an aromatic polyamide material.

### RELATED APPLICATIONS

This application claims priority to U.S. Provisional Application No. 63/481,463, filed on Jan. 25, 2023 and titled, “Fibrous Aramid Reinforced Catheter,” which is hereby incorporated by reference in its entirety.

### TECHNICAL FIELD

The present disclosure relates generally to medical devices. More specifically, in some embodiments, the present disclosure relates to vascular prosthesis deployment devices, including deployment devices for self-expanding and expandable vascular prostheses such as stents and stent-grafts.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 is a perspective view of an embodiment of a deployment device.

FIG. 2 is a cross-sectional view of a portion of the deployment device of FIG. 1.

FIG. 3A is a perspective view of a ratchet slide component of the deployment device of FIGS. 1 and 2.

FIG. 3B is a cross-sectional view of the ratchet slide of FIG. 3A.

FIG. 4 is a side view of a carrier component of the deployment device of FIGS. 1 and 2.

FIG. 5 is a cross-sectional view of another portion of the deployment device shown in FIGS. 1 and 2.

FIG. 6 is a cross-sectional view of yet another portion of the deployment device shown in FIGS. 1 and 2.

FIG. 7 is a front view of the deployment device of FIG. 1, illustrating certain cross-sectional planes described herein.

FIG. 8 is a perspective view of a safety member of the deployment device of FIG. 1.

FIG. 9 is a side view of a portion of the delivery catheter assembly of the deployment device of FIG. 1.

FIG. 10 is a side view of another portion of the delivery catheter assembly of the deployment device of FIG. 1.

FIG. 11 is a perspective view of a portion of an outer sheath of the delivery catheter assembly of the deployment device of FIG. 1.

FIG. 12 is a perspective view of a portion of an embodiment of a braided reinforcement structure.

FIG. 12A is a cross-sectional view of the braided reinforcement structure through section line 12A-12A of FIG. 12.

FIG. 12B is a cross-sectional view of the braided reinforcement structure through section line 12B-12B of FIG. 12.

FIG. 13 is perspective view of a portion of another embodiment of a braided reinforcement structure.

### DETAILED DESCRIPTION

Deployment devices may be configured to deliver a medical appliance to a location within a patient's body and deploy the medical appliance within the patient's body. Though specific examples recited herein may refer to deployment of devices within the vasculature, analogous concepts and devices may be used in various other locations within the body, including for placement and deployment of medical appliances in the gastrointestinal tract (including, for example, within the esophagus, intestines, stomach, small bowel, colon, and biliary duct); the respiratory system (including, for example, within the trachea, bronchial tubes, lungs, nasal passages, and sinuses); or any other location within the body, both within bodily lumens (for example, the ureter, the urethra, and/or any of the lumens discussed above) and within other bodily structures.

Furthermore, though specific examples herein may refer to deployment of vascular prostheses such as stents, deployment of a wide variety of medical appliances are within the scope of this disclosure, including stents, stent-grafts, shunts, grafts, and so forth. Additionally, the deployment device disclosed herein may be configured to deliver and deploy self-expanding medical appliances, including stents configured to expand within a bodily lumen upon deployment.

As used herein, delivery of a medical appliance generally refers to placement of a medical appliance in the body, including displacement of the appliance along a bodily lumen to a treatment site. For example, delivery includes displacement of a crimped stent along a vascular lumen from an insertion site to a treatment location. Deployment of a medical appliance refers to placement of the medical appliance within the body such that the medical appliance interacts with the body at the point of treatment. For example, deployment includes releasing a crimped or otherwise constrained self-expanding stent from a deployment device such that the stent expands and contacts a lumen of the vasculature.

Deployment devices within the scope of this disclosure may be configured to incrementally deploy a medical appliance. Incremental deployment may facilitate desired placement of the medical appliance due to the degree of control afforded a practitioner during deployment. A practitioner may for example, desire to deploy a portion of a stent, make adjustments to placement within the vasculature or confirm the location of the stent, prior to deploying the remaining portion of the stent. Such processes may be iterative, with a practitioner deploying a portion of a stent, confirming placement, deploying an additional portion, again confirming placement, and so forth until the stent is fully deployed.

Deployment devices within the scope of this disclosure may be configured to provide visual, audible, tactile, or other feedback relating to the degree to which a medical appliance has been deployed. Multiple types of feedback may enhance a practitioner's level of control over the procedure due to the multiple indications regarding location or degree of deployment of the medical appliance.

Moreover, deployment devices within the scope of this disclosure may provide a degree of mechanical advantage during deployment, for example, through the use of levers to decrease the force used to deploy a device. Mechanical advantage may thus increase a user's comfort and level of control during use. Still further, deployment devices within the scope of this disclosure may be ergonomically designed, presenting an actuation input disposed such that a practitioner can directly engage and utilize the device, without repositioning his or her hand or body. Deployment devices within the scope of this disclosure may also be configured for one-handed actuation and may be configured for ambidextrous use.

It will be readily understood that the components of the embodiments as generally described and illustrated in the figures herein could be arranged and designed in a wide variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “connected to” and “coupled to” refer to any form of interaction between two or more entities, including mechanical, electrical, magnetic, electromagnetic, fluidic, and thermal interaction. Two components may be coupled to each other even though they are not in direct contact with each other. For example, two components may be coupled to each other through an intermediate component.

The directional terms “proximal” and “distal” are used herein to refer to opposite locations on a medical device. The proximal end of the device is defined as the end of the device closest to the practitioner when the device is in use by the practitioner. The distal end is the end opposite the proximal end, along the longitudinal direction of the device, or the end furthest from the practitioner.

Again, though the embodiments specifically described below may reference a stent deployment device specifically, the concepts, devices, and assemblies discussed below may be analogously applied to deployment of a wide variety of medical appliances in a wide variety of locations within the body.

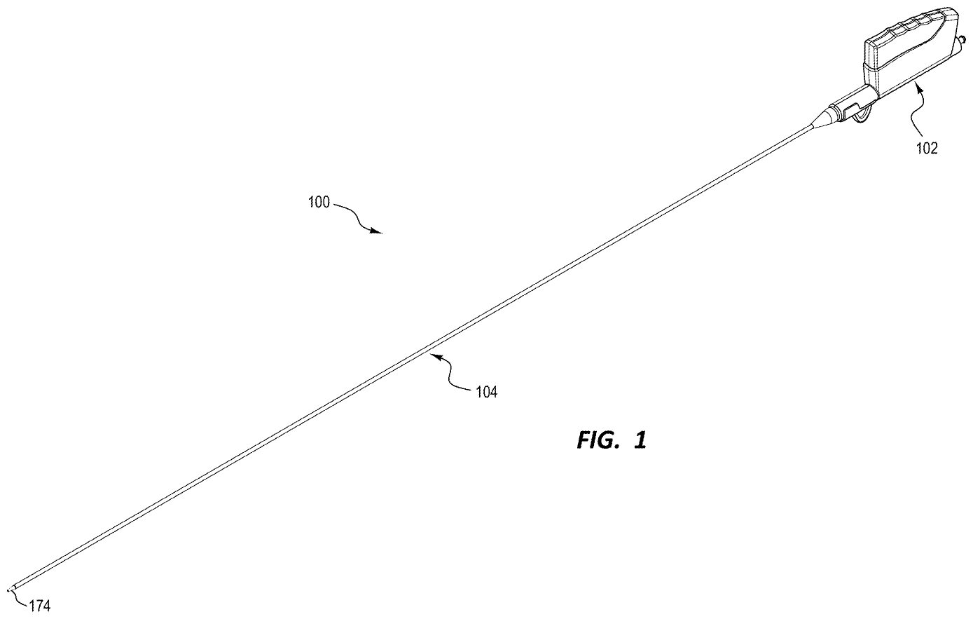

FIG. 1 is a perspective view of a deployment device 100. The deployment device 100 comprises a handle assembly 102 adjacent the proximal end of the deployment device 100. An elongate delivery catheter assembly 104 extends distally from the handle assembly 102 to a distal tip or delivery tip 174. The handle assembly 102 may provide a proximal user input, with one or more components configured to allow a practitioner to deploy or otherwise manipulate a stent disposed within the delivery catheter assembly 104.

In use, the handle assembly 102 may be disposed outside of a patient's body, while the delivery catheter assembly 104 is advanced to a treatment location within the patient's body. For example, the delivery catheter assembly 104 may be advanced from an insertion site (such as, for example, a femoral or jugular insertion site) to a treatment location within the vasculature. As further detailed below, the delivery catheter assembly 104 may be configured to be advanced through bends, turns, or other structures within the anatomy of the vasculature. Again, as detailed below, a stent may be disposed within a portion of the delivery catheter assembly 104 such that a practitioner may deploy the stent from a distal end of the delivery catheter assembly 104 through manipulation of one or more components of the handle assembly 102.

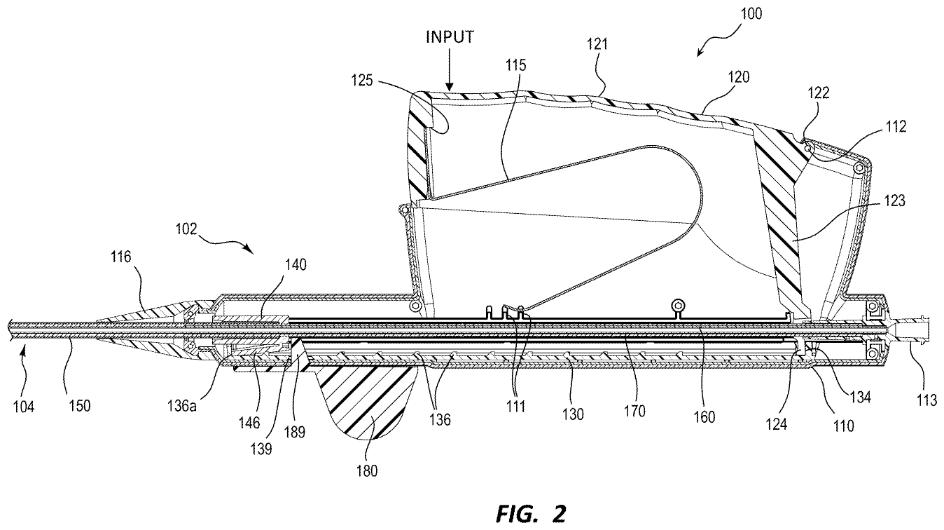

FIG. 2 is a cross-sectional view of a portion of the deployment device 100 of FIG. 1. Specifically, FIG. 2 is a side view of a portion of the deployment device 100 of

FIG. 1, taken through a cross-sectional plane extending vertically and intersecting a longitudinal axis of the deployment device 100, when the deployment device 100 is positioned as shown in FIG. 1. The longitudinal axis of the deployment device 100 extends along the center of the delivery catheter assembly 104, including along the center of components of the delivery catheter assembly 104 which overlap with the handle assembly 102, such as the intermediate sheath 160, as shown in FIG. 2.

As the handle assembly 102 is configured to be grasped or otherwise manipulated by a user and the delivery catheter assembly 104 is configured to extend to a treatment location within a patient's body, along the longitudinal axis, the delivery catheter assembly 104 extends in a distal direction away from the handle assembly 102. The proximal direction is opposite, correlating to a direction defined along the longitudinal axis, extending from the distal tip 174 toward the handle assembly 102.

FIG. 2 depicts various internal components of the handle assembly 102, exposed by the cross-sectional view. A portion of the delivery catheter assembly 104 is also shown extending from the handle assembly 102. The handle assembly 102 comprises a housing 110. The housing 110 surrounds certain components of the handle assembly 102, as shown, providing a grip surface for a practitioner.

The housing 110 is operably coupled to an actuator 120. Manipulation of the actuator 120 with respect to the housing 110 may be configured to deploy the stent, as further detailed below. In the depicted embodiment, the actuator 120 is rotatably coupled to the housing 110 by a pin 112. The pin 112 extends from the housing 110 and may be integrally formed with one or more other portions of the housing 110. As shown, the pin 112 extends through a pin aperture 122 in the actuator 120.

Other arrangements for operably coupling the actuator 120 and the housing 110 are within the scope of this disclosure. For example, the pin 112 may be integral with a portion of the actuator 120 and may be received in an opening, sleeve, or aperture formed in the housing 110. Other types of designs of rotatable couplings, including a separate coupling component such as a hinge are within the scope of this disclosure. Still further, a compliant mechanism, such as a deformable flange, may be utilized to rotatably couple the actuator 120 and the housing 110, including compliant couplings integrally formed with the actuator 120, the housing 110, or both. Moreover, it is within the scope of this disclosure to slidably couple an actuator (such as actuator 120) to a housing (such as housing 110). Configurations wherein the actuator 120 is manipulated through rotation, translation, or other displacement relative to the housing 110 are all within the scope of this disclosure.

The actuator 120 comprises an input portion 121 extending from the aperture 122. In the depicted embodiment, the input portion 121 comprises a surface, at least partially exposed with respect to the housing 110. In operation, a user may manipulate the actuator 120 by exerting a force on the input portion 121, illustrated by the arrow labeled “input” in FIG. 2, displacing the input portion 121 generally toward the longitudinal axis of the deployment device 100 and causing the actuator 120 to rotate about the pin 112 with respect to the housing 110. Displacement of the actuator 120 due to a force such as illustrated by the arrow labeled “input” corresponds to “depression” of the actuator 120 or “depression of the actuator 120 with respect to the housing 110.”

The actuator 120 may further comprise a transfer arm 123 extending from the pin aperture 122. The transfer arm 123 may be rigidly coupled to the input portion 121, including embodiments wherein both the transfer arm 123 and the input portion 121 are integrally formed with the rest of the actuator 120. The transfer arm 123 extends to a ratchet slide engaging portion 124. Depression of the input portion 121, in the direction shown by the arrow labeled “input” displaces the transfer arm 123 as the actuator 120 is rotated about the pin 112.

Depression of the input portion 121 thus causes displacement of the ratchet slide engaging portion 124 with respect to the housing 110. This displacement of the ratchet slide engaging portion 124 can be understood as rotation about the pin 112 having a proximal translation component and a vertical translation component, as rotation of the input portion 121 in the direction indicated by the arrow labeled “input” will displace (with respect to the housing 110) the ratchet slide engaging portion 124 both proximally and vertically.

A spring 115 may be disposed between the actuator 120 and the housing 110. The spring 115 may be configured to resist displacement of the actuator 120 in the direction indicated by the arrow labeled “input” and may be configured to return the actuator to the relative position shown in FIG. 2 after it has been depressed by a user. When the handle assembly 102 is unconstrained, the spring 115 may thus maintain (or return to) the relative position of the actuator 120 with respect to the housing 110 as shown in FIG. 2.

In the illustrated embodiment, the spring 115 engages with a spring ledge 125 of the actuator 120 and spring protrusions 111 of the housing 110. The spring protrusions 111 may provide a bearing surface for the spring 115 offset from movable internal components of the handle assembly 102 (such as a carrier 140 further detailed below). Though three spring protrusions 111 are shown in the depicted embodiment, more or fewer protrusions, or use of other features such as ridges, ledges, shoulders, and so forth are within the scope of this disclosure.

The depicted embodiment comprises a leaf spring 115. Other biasing elements, such as coil springs, piston assemblies, compliant mechanisms, and so forth are likewise within the scope of this disclosure. In some instances, a compliant portion of one or both of the housing 110 and actuator 120 may provide a biasing force analogous to that provided by the spring 115. Leaf springs, such as spring 115, may be configured to provide a relatively constant biasing force notwithstanding compression of the spring 115 as the actuator 120 is rotated or depressed with respect to the housing 110.

As the actuator 120 is depressed with respect to the housing 110, the spring 115 compresses and the ratchet slide engaging portion 124 is displaced as described above. Again, the displacement of the ratchet slide engaging portion 124 with respect to the housing 110 can be understood as having a proximal component and a vertical component.

The ratchet slide engaging portion 124 may be operably coupled to a ratchet slide 130 such that displacement of the ratchet slide engaging portion 124 likewise displaces the ratchet slide 130. The ratchet slide 130 may be constrained such that the ratchet slide 130 is configured only for proximal or distal displacement with respect to the housing 110. Thus, operable coupling of the ratchet slide engaging portion 124 to the ratchet slide 130 may allow for sliding interaction between the ratchet slide engaging portion 124 and the ratchet slide 130 such that only the proximal or distal component of the displacement of the ratchet slide engaging portion 124 is transferred to the ratchet slide 130. Stated another way, the ratchet slide 130 may be displaced in a direction parallel to the longitudinal axis of the deployment device 100 while the input displacement may be at an angle to the longitudinal axis of the deployment device 100. It is noted that, in the configuration shown in FIG. 2, a safety member 180 may prevent proximal displacement of the ratchet slide 130. The safety member 180, including removal thereof, is discussed in more detail below. Discussion herein relating to displacement of the ratchet slide 130 and related components may thus be understood as disclosure relevant to a configuration of the handle assembly 102 in which the safety member 180 has been removed.

As the actuator 120 is depressed with respect to the housing 110, the ratchet slide 130 may thus be proximally displaced with respect to the housing 110. One or both of the ratchet slide 130 and actuator 120 may also interact with the housing 110 such that there is a positive stop to arrest the depression of the actuator 120 and/or proximal displacement of the ratchet slide 130. This positive stop may be an engaging ledge, shoulder, lug, detent, or other feature coupled to the housing 110, including features integrally formed on the housing 110.

A full stroke of the actuator 120 may thus correspond to displacement from the unconstrained position shown in FIG. 2, to the positive stop caused by interaction with the housing 110 when the actuator 120 is depressed. Release of the actuator 120 following a full or a partial stroke may then result in a return of the actuator 120 to the unconstrained state, due to the biasing force provided by the spring 115. The unconstrained state shown in FIG. 2 refers to lack of constraint due to user input. In this state, the spring 115 may be partially compressed, and interaction between the actuator 120 and the housing 110 may prevent rotation of the actuator 120 about the pin 112 in the opposite direction to depression of the actuator 120, or the return direction. In other words, interaction between the actuator 120 and the housing 110 (or features of the housing 110) may create a positive stop to the return motion of the actuator 120 as well.

Referring to both FIGS. 1 and 2, the actuator 120 and the housing 110 may be coupled such that pinching of external materials (such as a practitioner's hand or a surgical drape) is minimized when the actuator 120 is depressed or returned. For instance, the actuator 120 may comprise a shell configured to mate with, and slide into, the housing 110. Though the components may slide and rotate with respect to each other, the interface of the components may be sufficiently close and/or smooth to minimize pinching or other engagement of external materials. This close and/or smooth interface may refer to interaction at the edges of the actuator 120 as it is displaced into the housing 110 and/or to interaction at the portion of the actuator 120 near the pin 112, as the actuator 120 returns to the unconstrained position.

As also shown in FIGS. 1 and 2, the input portion 121 of the actuator 120 may also comprise ridges or other features to facilitate handling or gripping of the actuator 120 during use.

Referring again to FIG. 2, the ratchet slide 130 may thus be proximally displaced during depression of the actuator 120. Again, such displacement may correspond to a configuration in which the safety member 180 shown in FIG. 2 has been removed. Proximal displacement of the ratchet slide 130 may also proximally displace the carrier 140 due to interaction between one or more carrier engaging ratchet lugs 136 on the ratchet slide 130 and a ratchet slide engaging arm 146 coupled to the carrier 140.

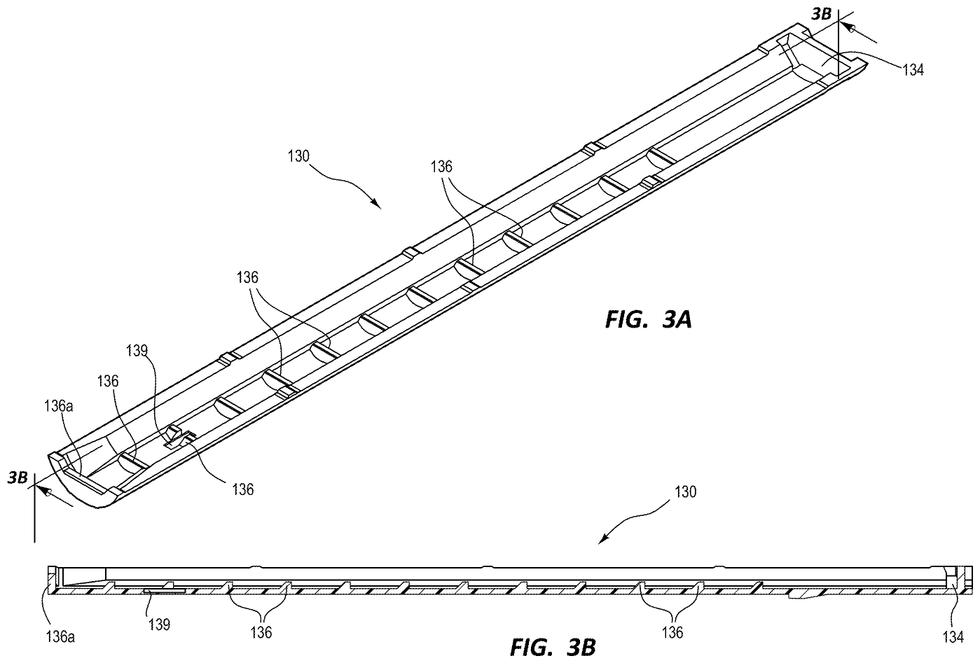

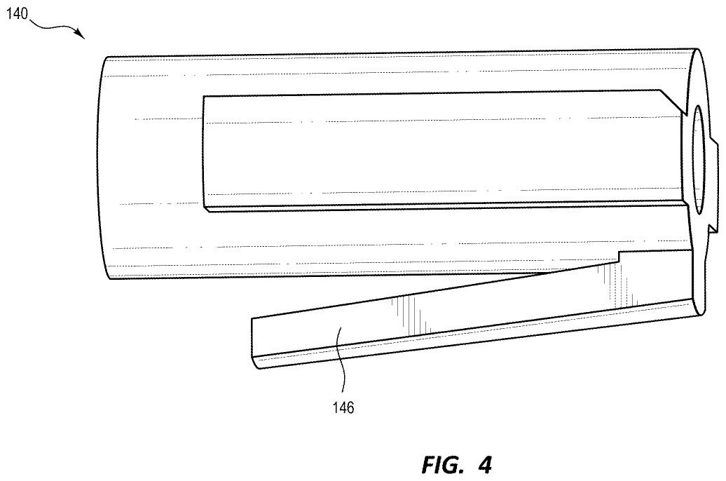

FIG. 3A is a perspective view of the ratchet slide 130 of the deployment device 100 of FIGS. 1 and 2. FIG. 3B is a cross-sectional view of the ratchet slide 130 of FIG. 3A, taken through a vertical plane disposed along a longitudinal centerline of the ratchet slide 130. When the ratchet slide 130 is disposed within the handle assembly 102 of FIG. 2, this cross-sectional plane would intersect the longitudinal axis of the deployment device 100.

As shown in FIGS. 2, 3A, and 3B, the ratchet slide 130 may comprise a plurality of carrier engaging ratchet lugs 136. The carrier engaging ratchet lugs 136 may be spaced at even intervals along the longitudinal direction of the ratchet slide 130. In the figures, exemplary carrier engaging ratchet lugs are denoted with reference numeral 136, while the distal most carrier engaging ratchet lug, disposed at the distal end of the ratchet slide 130 is denoted with reference numeral 136a.

The ratchet slide 130 further comprises a ratchet slide safety opening 139 and an actuator engaging opening 134. These features are discussed in more detail below.

As noted above, interaction between the ratchet slide engaging portion 124 of the actuator 120 and the ratchet slide 130 may proximally displace the ratchet slide 130 with respect to the housing 110. Engagement between the carrier 140 and one of the carrier engaging ratchet lugs 136 may also proximally displace the carrier 140 as the ratchet slide 130 is proximally displaced with respect to the housing 110. In the configuration of FIG. 2, the ratchet slide engaging arm 146 of the carrier 140 is engaged with the distal most carrier engaging ratchet lug 136a.

FIG. 4 is a side view of the carrier 140 of the deployment device 100 of FIGS. 1 and 2. As shown in FIG. 4, the ratchet slide engaging arm 146 extends radially away from a longitudinal axis of the carrier 140. When the carrier 140 is disposed within the handle assembly 102 of FIG. 2, the longitudinal axis of the carrier 140 is disposed along the longitudinal axis of the deployment device 100.

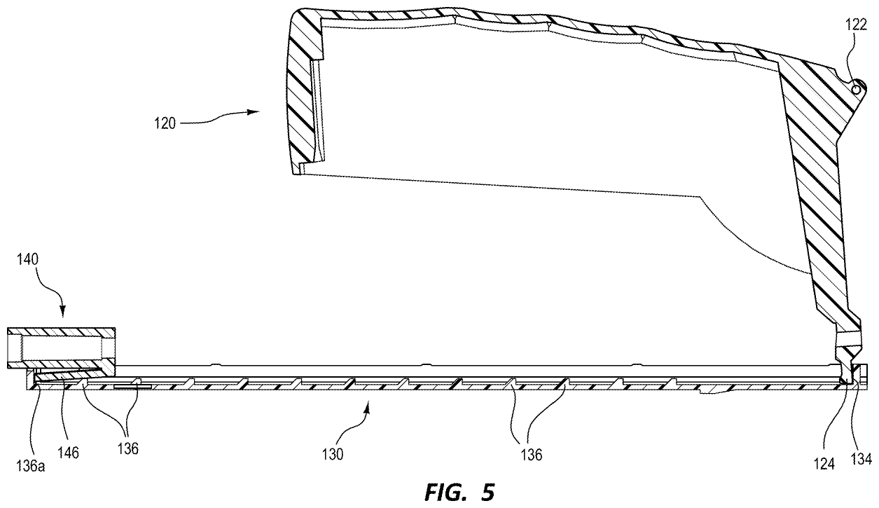

FIG. 5 is a cross-sectional view of a portion of the deployment device 100 shown in FIGS. 1 and 2. Specifically, the actuator 120, ratchet slide 130, and carrier 140 are shown in FIG. 5, in the same relative positions, and along the same cross-sectional plane as in FIG. 2.

Referring to FIGS. 2-5, during depression of the actuator 120 with respect to the housing 110, the actuator 120 rotates around the pin aperture 122. This rotation causes displacement of the ratchet slide engaging portion 124 of the actuator 120. The component of this displacement correlating to proximal displacement of the ratchet slide engaging portion 124 also proximally translates the ratchet slide 130 due to interaction between the ratchet slide engaging portion 124 of the actuator 120 and the actuator engaging opening 134 of the ratchet slide 130. Stated another way, the walls or faces that define the actuator engaging opening 134 may contact the ratchet slide engaging portion 124 such that the ratchet slide 130 is displaced when the actuator 120 is displaced.

Proximal displacement of the ratchet slide 130 also proximally displaces the carrier 140 due to interaction between the carrier engaging ratchet lugs 136 and the ratchet slide engaging arm 146. In the depicted embodiment, a distal surface of the ratchet slide engaging arm 146 is in contact with a proximal face of the distal most carrier engaging ratchet lug 136a. This contact exerts proximal force on the distal surface of the ratchet slide engaging arm 146, displacing the carrier 140 in a proximal direction. Accordingly, the ratchet slide 130 and carrier 140 will move proximally until the actuator 120 reaches the end of the stroke.

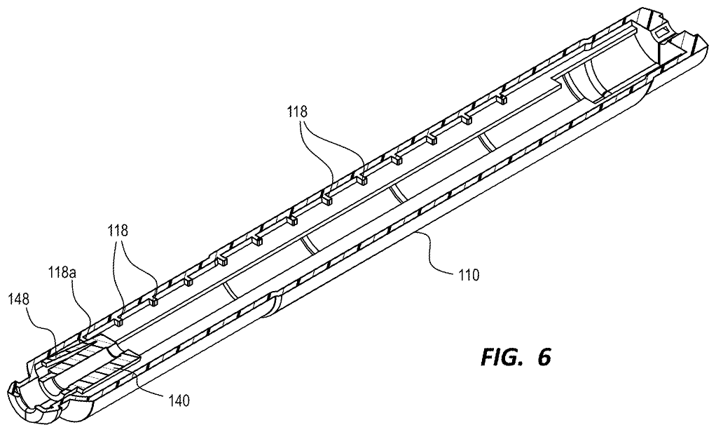

FIG. 6 is a cross-sectional view of the housing 110 and the carrier 140 in the same relative positions shown in FIG. 2. The cross-sectional plane of FIG. 6 extends along the longitudinal axis of the deployment device; however, the cross-sectional plane of FIG. 6 extends horizontally, orthogonal to the cross-sectional planes of FIGS. 2, 3B, and 5.

As shown in FIG. 6, the carrier 140 comprises a housing engaging arm 148 extending radially away from a longitudinal axis of the carrier 140. The housing 110 comprises a plurality of carrier engaging housing lugs 118. In FIG. 6, exemplary carrier engaging housing lugs are denoted by reference numeral 118, with the distal most carrier engaging housing lug denoted by reference numeral 118a.

Referring to FIGS. 2-6, as interaction between the actuator 120, ratchet slide 130, and carrier 140 displaces the carrier 140 with respect to the housing 110 (as shown and described above), the housing engaging arm 148 (shown in FIG. 6) of the carrier 140 will deflect radially inward due to contact with one of the carrier engaging housing lugs 118. For example, from the position shown in FIG. 6, as interaction between the distal most carrier engaging ratchet lug 136a and the ratchet slide engaging arm 146 of the carrier 140 draws the carrier 140 proximally, the distal most carrier engaging housing lug 118a causes the housing engaging arm 148 to displace radially inward. The housing engaging arm 148 will continue to deflect radially inward until the distal end of the housing engaging arm 148 is positioned proximal of the distal most carrier engaging housing lug 118a, at which point the housing engaging arm 148 will return to the radially outward configuration shown in FIG. 6. The point at which the housing engaging arm 148 moves proximally of the distal most carrier engaging housing lug 118a, may correspond to the stroke of the actuator 120, such that engagement between the housing engaging arm 148 and the next carrier engaging housing lug 118 (moving in a proximal direction) occurs at the end of the stroke, which may correspond to contact between the ratchet slide 130 and/or actuator 120 and a positive stop on the housing 110 defining the end of the stroke.

As the actuator 120 is released following the stroke, interaction between the spring 115, the housing 110, and the actuator 120 will return the actuator 120 to the unconstrained position (the position shown in FIG. 2) as discussed above. Corresponding rotation of the actuator 120 about the pin aperture 122 will thus correlate to displacement of the ratchet slide engaging portion 124, including a component of displacement in the distal direction. Interaction between the ratchet slide engaging portion 124 and the actuator engaging opening 134 will then correlate to distal displacement of the ratchet slide 130. Thus, when the actuator 120 is released at the end of a stroke, the actuator 120, the spring 115, and the ratchet slide 130 return to the same positions relative to the housing as shown in FIG. 2.

As the actuator 120 returns to the unconstrained position, however, interaction between the housing engaging arm 148 and the carrier engaging housing lug 118 prevents distal displacement of the carrier 140. Specifically, the distal surface of the housing engaging arm 148 will be in contact with a proximal facing surface of a carrier engaging housing lug 118, the interaction preventing the carrier 140 from returning to the pre-stroke position. In the exemplary stroke discussed above, the distal most carrier engaging housing lug 118a displaced the housing engaging arm 148 during the stroke, and the housing engaging arm 148 engaged with the distal most carrier engaging housing lug 118a following the stroke. Subsequent strokes move the carrier 140 along the plurality of carrier engaging housing lugs 118 in a proximal direction.

As the actuator 120 returns to the unconstrained state, radially inward displacement of the ratchet slide engaging arm 146 of the carrier 140 allows the ratchet slide 130 to move distally with respect to the carrier 140, as engagement between the carrier 140 and the carrier engaging housing lugs 118 arrest distal displacement of the carrier 140.

Referring to FIGS. 2-6, with particular reference to the view of FIG. 5, distal displacement of the ratchet slide 130 with respect to the carrier 140 creates interaction between the carrier engaging ratchet lugs 136 and the ratchet slide engaging arm 146 causing the ratchet slide engaging arm 146 to displace radially inward. The proximal facing surface of the carrier engaging ratchet lugs 136 may be angled to facilitate this interaction. In the exemplary stroke discussed above, engagement between the distal most carrier engaging ratchet lug 136a displaced the carrier 140 in a proximal direction; during the return of the actuator 120, the next carrier engaging ratchet lug 136 (in a proximal direction) causes the radially inward displacement of the ratchet slide engaging arm 146 until the ratchet slide engaging arm 146 is proximal of the carrier engaging ratchet lug 136. At that point the ratchet slide engaging arm 146 returns to a radially outward position (analogous to that shown in FIG. 5) though the distal surface of the ratchet slide engaging arm 146 is now engaged with a proximal face of the next carrier engaging ratchet lug 136 (again in a proximal direction). Displacement of the ratchet slide 130 sufficient to move to engagement with a subsequent carrier engaging ratchet lug 136 may correspond with the magnitude of ratchet slide 130 displacement corresponding to a return of the actuator 120. Subsequent returns of the actuator 120 following strokes move the ratchet slide 130 such that the plurality of carrier engaging ratchet lugs 136 may serially engage the carrier 140, stroke after stroke.

Accordingly, as described above, depressing the actuator 120 for a full stroke, then allowing the actuator 120 to return to the unconstrained position, displaces the carrier 140 with respect to the housing 110 in discrete increments, corresponding to the distance between adjacent carrier engaging housing lugs 118 along the longitudinal direction. Interaction of the actuator 120 and positive stops associated with the housing 110, carrier arms (e.g., ratchet slide engaging arm 146 and housing engaging arm 148), and lugs (e.g., carrier engaging housing lugs 118 and carrier engaging ratchet lugs 136) may also combine to give a user tactile and audible feedback as the carrier 140 is incrementally displaced. Further, one or more opening in the housing 110 may allow a user to observe the relative position of the carrier 140 providing further feedback as to carrier 140 position.

As detailed below, the relative position of the carrier 140 with respect to the housing 110 may correlate to the degree of deployment of a stent from the deployment device 100. Thus, visual, audible, and tactile feedback as to the position of the carrier 140 provides a user with information regarding stent deployment during use of the deployment device 100. This information may correlate to increased control during deployment as the practitioner quickly and intuitively can surmise the degree of stent deployment.

As outlined above, tactile and/or audible feedback result from the interactions of the carrier 140, ratchet slide 130, housing 110, and/or actuator 120. For example, as the ratchet slide engaging arm 146 or housing engaging arm 148 of the carrier 140 deflects radially inward then return outward, there may be an audible and/or tactile response.

The device may be configured for visual feedback of, or relating to, the relative deployment of a stent. For example, in some embodiments, the housing 110 may comprise viewing windows to allow a practitioner to observe the position of the carrier 140 relative to the housing 110. Further, indicia on the housing 110 may correlate the position of the carrier 140 to the degree of deployment of a stent.

The increments of displacement of the carrier 140 may correlate to standard stent lengths or units of measure. For example, many stents are sized in 1 cm increments. Configuration of the increments of displacement on the carrier 140 in 1 cm increments would thus directly correlate with stent length at a 1:1 ratio. Any other ratio, including embodiment wherein a stroke correlates to a greater length (such as 2, 3, 4, or 5 cm) or a lesser length (such as 0.01, 0.1, 0.25, 0.5, or 0.75 cm) are likewise within the scope of this disclosure.

In some embodiments, interaction between the carrier 140, the ratchet slide 130, the housing 110, and/or the actuator 120 may comprise additional carrier engaging ratchet lugs 136 and/or carrier engaging housing lugs 118. For example, the carrier engaging ratchet lugs 136 may be spaced to enable semi-continuous ratcheting of the ratchet slide 130 with respect to the actuator 120 and/or the housing 110.

The deployment device 100 may be configured as a universal device operable with various stent lengths. In some embodiments a practitioner may directly equate the number of strokes needed to deploy a stent with the length of the stent loaded in the deployment device 100 (such as four strokes for a four centimeter stent). Further, a single design of deployment device 100 may be utilized with various lengths of stents, with a maximum length related to the maximum length of travel of the carrier 140.

The nature of depression of the actuator 120 may facilitate one-handed operation and may be ergonomically designed. First, a practitioner need only grip the deployment device with one hand to depress the actuator, leaving a second hand free for other therapy needs. Further, the direction with which the deployment device is gripped, with the practitioner's hand extending laterally away from the longitudinal axis of the deployment device and the lateral direction of depression, as opposed, for example, to longitudinal gripping to actuate, may be ergonomically desirable. Lateral gripping and input may more readily present the deployment device 100 for use when the delivery catheter assembly 104 is disposed within a patient's body, not requiring the practitioner to move to an awkward stance with respect to other therapy tools. Further, the input portion 121 of the actuator 120 may provide additional surface for a practitioner to grip, facilitating use of a greater portion of a practitioner's hand for actuation, as compared to a finger trigger or similar actuation mechanism.

The incremental displacement of the carrier 140 may further facilitate partial deployment of a stent, allowing a practitioner to deploy the stent in increments, potentially adjusting or confirming the position of the stent between these increments.

Still further, the deployment device 100 may be configured for use with either the right or left hand, or gripped with the fingers or palm in contact with the actuator 120 without changing the design of the deployment device 100. These features may further increase user comfort and control. Viewing windows in the housing 110 to confirm the position on the carrier 140 may be located on one or both sides of the housing 110 and may be associated with indicia correlating to stent length or other factors.

Moreover, the relative lengths of the input portion 121 and transfer arm 123 of the actuator 120 may be configured to provide mechanical advantage when deploying a stent. This may increase comfort and control during use. The ratio of the length of the input portion 121—from its distal end to the pin aperture 122—to the length of the transfer arm 123—from the pin aperture 122 to the ratchet slide engaging portion 124—may be greater than or equal to 1.5:1, including 2:1, 2.5:1, 3:1, 3.5:1 or greater. This ratio correlates to the mechanical advantage provided by the device. In some instances the mechanical advantage provided may be 1.5:1, 2:1, 2.5:1, 3:1, 3.5:1 or greater. Stated another way, the ratio of length of travel of the input portion 121 to the corresponding length of travel of the ratchet slide engaging portion 124 may be 1.5:1, 2:1, 2.5:1, 3:1, 3.5:1 or greater. Accordingly, the input force applied against the input portion 121 may result in a greater force exerted by the ratchet slide engaging portion 124 on the ratchet slide 130. The ratio of the force exerted on the ratchet slide 130 to the input force may be 1.5:1, 2:1, 2.5:1, 3:1, 3.5:1 or greater.

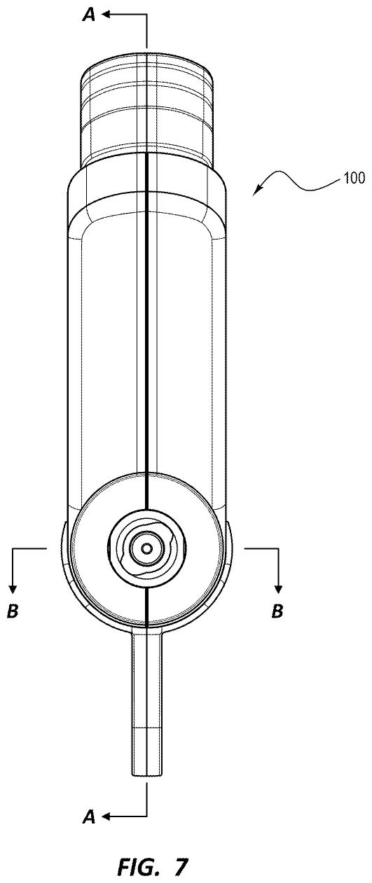

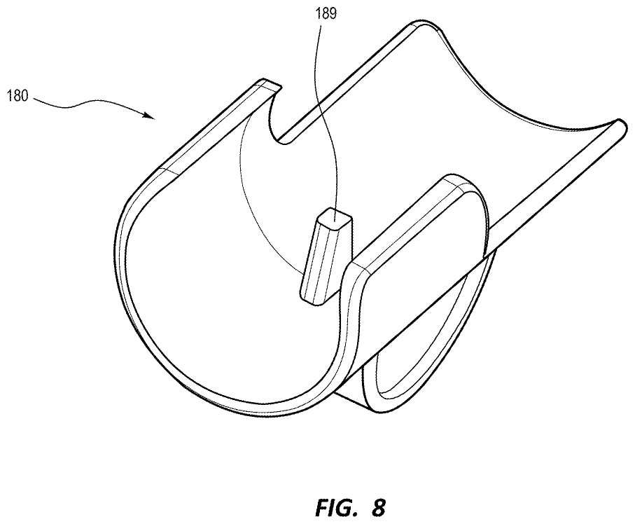

FIG. 7 is a front view of the deployment device 100, illustrating two cross-sectional planes. Specifically, plane A-A extends vertically along the longitudinal axis of the deployment device 100 viewing the exposed components in a right to left direction. Plane A-A corresponds to the cross-sectional plane of FIGS. 2, 3B, and 5. Plane B-B also extends from the longitudinal axis of the deployment device 100, though Plane B-B extends horizontally therefrom. Plane B-B corresponds to the cross-sectional plane of FIG. 6, and is viewed from a top to bottom direction. The longitudinal axis of the deployment device 100 is in both planes A-A and B-B, with the line defined as the intersection between these planes being the same line as the longitudinal axis as referenced herein.

Additionally, as stated above, the deployment device 100 may comprise a safety member 180. FIG. 8 is a perspective view of the safety member 180 of the deployment device 100. The safety member 180 may be configured with a circular or partially circular opening configured to snap onto an outside surface of a portion of the deployment device 100 and a grip member 181 configured to be gripped by a practitioner when removing the safety member 180. Referring to both FIG. 2 and FIG. 8, the safety member 180 may comprise a safety lug 189 that extends through a ratchet slide safety opening (139 of FIG. 3A) and a similar safety opening in the housing 110 (not shown). When the safety lug 189 is disposed within these openings, the safety lug 189 may prevent proximal displacement of the carrier 140 and the ratchet slide 130, thus preventing inadvertent deployment of a stent. A practitioner may leave the safety member 180 in place during displacement of the delivery catheter assembly 104 to a treatment region. Due to interactions between the carrier 140, ratchet slide 130, and actuator 120, the safety member 180 likewise prevents displacement of the actuator 120 when the safety lug 189 extends through the openings.

In the depicted embodiment, the safety lug 189 extends through a bottom portion of the housing 110 and ratchet slide 130. In other embodiments, the safety lug 189 may extend through a top surface of the housing 110, interacting with the carrier 140 but not directly with the ratchet slide 130. Nevertheless, prevention of proximal displacement on the carrier 140 only, will also prevent displacement of the ratchet slide 130 and the actuator 120 due to the interaction between these elements.

In some embodiments, the safety member 180 may be tethered to the deployment device 100, or may comprise a sliding switch or other element operably coupled to the housing 110 or other components of the deployment device 100. In the depicted embodiment, the safety member 180 is removably coupled.

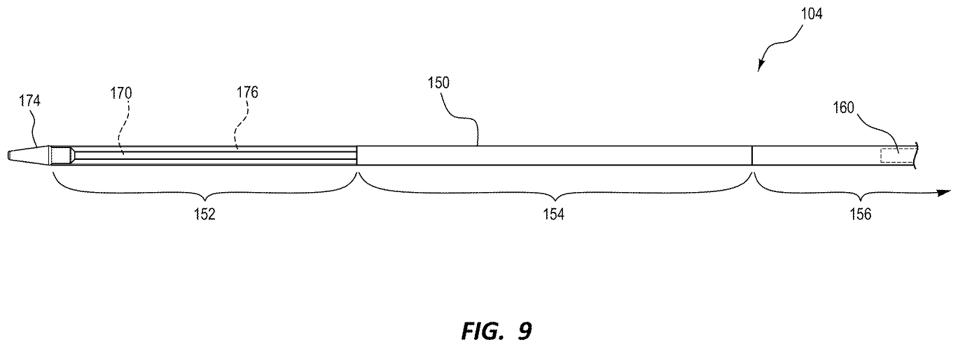

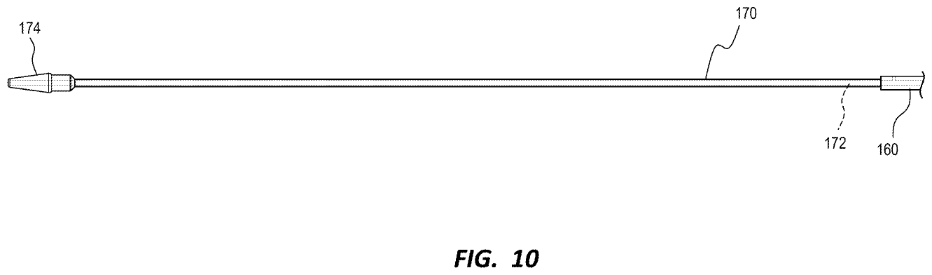

FIG. 9 is a side view of a portion of the delivery catheter assembly 104 of the deployment device 100. Specifically, FIG. 9 is a side view of a distal section of the delivery catheter assembly 104. FIG. 10 is a side view of the same longitudinal section of the delivery catheter assembly 104 as shown in FIG. 9; however, the outer sheath (150 of FIG. 9) has been removed to show other components.

Referring to FIGS. 1, 2, 9, and 10, the delivery catheter assembly 104 may be configured to deploy a stent as the deployment device 100 is manipulated, as discussed above. The delivery catheter assembly 104 may comprise an outer sheath 150, extending from the handle assembly 102. The outer sheath 150 may be fixedly coupled to the carrier 140. The delivery catheter assembly 104 may further comprise an intermediate sheath 160 and an inner sheath 170, both disposed within the outer sheath 150, and both fixedly coupled to the housing 110. Thus, proximal displacement of the carrier 140 with respect to the housing 110 will proximally displace the outer sheath 150 with respect to both the intermediate sheath 160 and the inner sheath 170.

The outer sheath 150 may comprise a shaft section 156 extending from the carrier 140 in a distal direction. At the distal end of the shaft section 156 the outer sheath 150 may comprise a flex zone 154 extending from the shaft section 156 in a distal direction. Finally, the outer sheath 150 may comprise a pod 152 extending from the flex zone 154 in a distal direction. (As shown in FIG. 9, the pod 152 may be transparent.)

The shaft section 156 of the outer sheath 150 may have a different stiffness and/or durometer than the flex zone 154 and/or the pod 152. The flexibility toward the distal end of the outer sheath 150 may improve trackability of the delivery catheter assembly 104 over a guidewire and may be less traumatic, while a stiffer shaft may be more kink resistant and/or transmit displacement and/or torque along the shaft section 156.

The pod 152 may be configured to retain a crimped or otherwise constrained stent. Removal of the pod 152 from the stent may allow the stent to self-expand, and thereby deploy. It is within the scope of this disclosure for the pod 152 to be any relative length, the flex zone 154 to be any relative length, and the shaft section 156 to be any relative length. Thus, in some instances, a constrained stent may be in one, two, or all three of these portions of the outer sheath 150. For example, in the illustrated embodiment, an annular space 176 (described further below) is configured to receive a crimped stent extending along the pod 152 as well as portions of the flex zone 154 and shaft section 156. In other embodiments, the annular space 176 may correlate just to the pod 152 segment, meaning the device is configured to retain a crimped stent only within the pod 152 segment.

The distal tip 174 of the delivery catheter assembly 104 may be coupled to and/or integrally formed with the inner sheath 170. A lumen 172 may extend along the inner sheath 170 from the proximal end of the deployment device 100 to the distal tip 174. A luer fitting 113 coupled to the housing 110 (as shown in FIG. 2) may be in communication with the lumen 172. A guidewire may thus extend through the luer fitting 113, through the lumen 172, and out of the distal tip 174. Further, fluid introduced into the luer fitting 113 may be utilized to flush the lumen 172.

The inner sheath 170 may be fixed to the housing 110, for example, at the proximal end of the inner sheath 170. An intermediate sheath 160, also fixed to the housing 110, may extend over a portion of the inner sheath 170. The intermediate sheath 160 and inner sheath 170 may or may not be directly fixed to each other. In some embodiments, the intermediate sheath 160 may be a close slip fit over the inner sheath 170.

The inner sheath 170 extends distally beyond a distal end of the intermediate sheath 160, creating an annular space 176 between the inner sheath 170 and the outer sheath 150 adjacent the distal tip 174, extending proximally to the distal end of the intermediate sheath 160. This annular space 176 may be configured to retain a crimped stent.

As the deployment device 100 is manipulated to incrementally displace the carrier 140 with respect to the housing 110, the outer sheath 150 is incrementally displaced proximally with respect to the inner sheath 170 and intermediate sheath 160. The distal end of the intermediate sheath 160 interacts with the proximal end of the stent, preventing the stent from being drawn back with the outer sheath 150. Thus, the stent is incrementally exposed, and allowed to self-expand and deploy.

The distal tip 174 may comprise a flexible material and may be configured to be atraumatic. The distal tip 174 may comprise nylons, including PEBAX® polyether block amides.

One, two, or all of the outer sheath 150, the intermediate sheath 160, and/or the inner sheath 170 may be configured with varying durometers or other properties along the length thereof. In some instances, the outer sheath 150 may be configured with a proximal section with a durometer between 72 and 100 on the Shore D scale or may be greater than 100 on the Shore D scale. A second portion of the outer sheath 150 may comprise a durometer of 63 on the Shore D scale, and a distal section with a durometer between 40 and 55 on the Shore D scale. Any of these values, or the limits of any of the ranges, may vary by 15 units in either direction. In some instances, the second portion will begin about six inches from the distal end of the outer sheath 150, and the distal section will begin about three inches from the distal end of the outer sheath 150. These sections may or may not correspond to the shaft section 156, the flex zone 154, and the pod 152 as described above. The intermediate sheath 160 may be configured with varying durometer zones within the same ranges of hardness and length.

Any of the inner sheath 170, intermediate sheath 160, and outer sheath 150 may have differing durometer or flex zones along their lengths, and these zones may overlap in various ways to create various stress/strain profiles for the overall delivery catheter assembly 104. Overlapping of such zones may reduce tendency to kink, including tendency to kink at transition zones. Further, the housing 110 may be coupled to a strain relief member 116 (as shown in FIG. 2).

Any of the outer sheath 150, the intermediate sheath 160, and the inner sheath 170 may be comprised of nylons, including PEBAX® polyether block amides. Further, during manufacture, any of these members may be configured with a low friction outer surface, including through “frosting” the materials, by blowing air across the material during extrusion, or by using additives during extrusion to reduce friction.

In some instances, during manufacture the distal tip 174 may be pulled into interference with the outer sheath 150, prestressing the inner sheath 170 in tension. This may reduce any effects of material creep or elongation during sterilization, keeping the distal tip 174 snugly nested with the outer sheath 150. Further, during manufacture, the interface zone between the outer sheath 150 and the carrier 140 may be configured with a tolerance zone, meaning the outer sheath 150 can be coupled to the carrier 140 at multiple points along an inside diameter of the carrier 140. This tolerance may enable manufacturing discrepancies or variations to be taken up during assembly to ensure a snug nest between the distal tip 174 and the outer sheath 150. The same tolerance fit may be applied to the inner sheath 170 and/or the intermediate sheath 160 wherein these members couple to the housing 110, including a fit zone along an inside diameter of the luer fitting 113.

In some instances, the outer sheath 150 may include indicia correlating to the degree to which a stent has been deployed. These indicia may correspond to the position of the outer sheath 150 with respect to the housing 110. For example, as the outer sheath 150 is drawn into the housing 110, different indicia are exposed and/or covered.

Further, in some instances, the deployment device 100 may be configured such that the outer sheath 150 may be distally displaced after the stent is deployed to nest the distal tip 174 in the outer sheath 150 during withdrawal of the deployment device 100 from a patient. Such configurations may include features of the handle assembly 102 that disengage the carrier 140 from one or more elements after stent deployment.

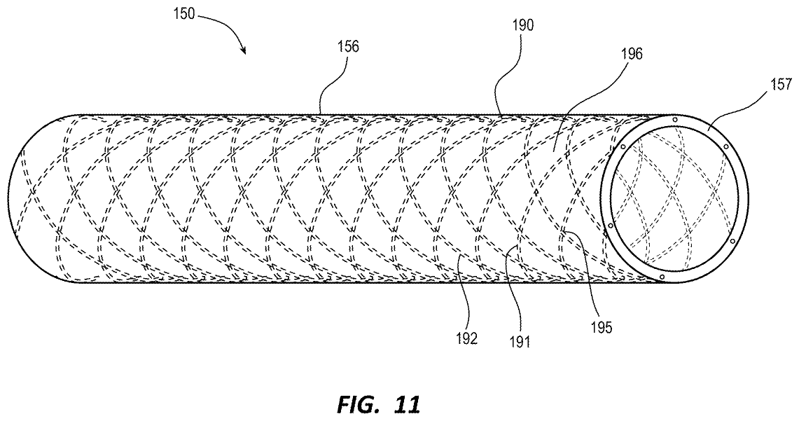

As illustrated in FIG. 11, the shaft section 156 of the outer sheath 150 may include a braided reinforcement structure 190 configured to increase kink resistance and reduce ovalization (i.e., deformation to a non-round or oval shape) when the outer sheath 150 is passed through a tortuous vessel. The braided reinforcement structure 190 may also increase resistance to elongation of the outer sheath 150 as it is incrementally displaced proximally to provide accuracy in the positioning of the deployed stent. In other embodiments, the flex zone 154 and the pod 152 may include the braided reinforcement structure 190. In the illustrated embodiment of the outer sheath 150 of FIG. 11, the braided reinforcement structure 190 is embedded within a circumferential wall 157. The braided reinforcement structure 190 extends the length of the shaft section 156. In another embodiment, the braided reinforcement structure 190 may extend partially along the length of the shaft section 156. The braided reinforcement structure 190 is surrounded by a material, wherein the braided reinforcement structure 190 is not exposed at an exterior surface and an interior surface of the wall 157. The material is disposed within a mesh 196 to restrict elongation of the braided reinforcement structure 190 when an axial tension load is applied to the shaft section 156. The material can be any suitable material to provide desired characteristics (e.g., stiffness, tensile strength, elongation) to facilitate placement and deployment of the stent at a desired location. For example, in some embodiments, the material may comprise pebax vestimide and/or other reflowable materials.

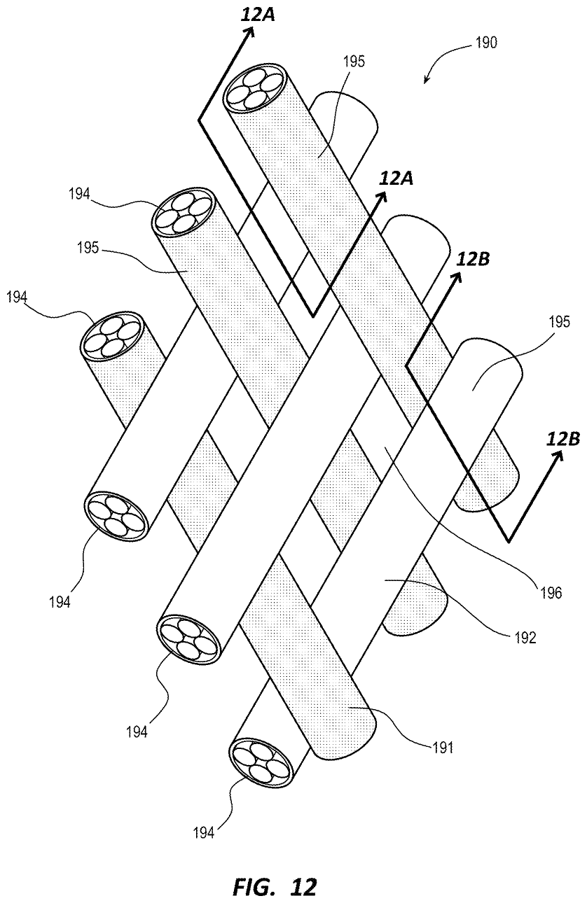

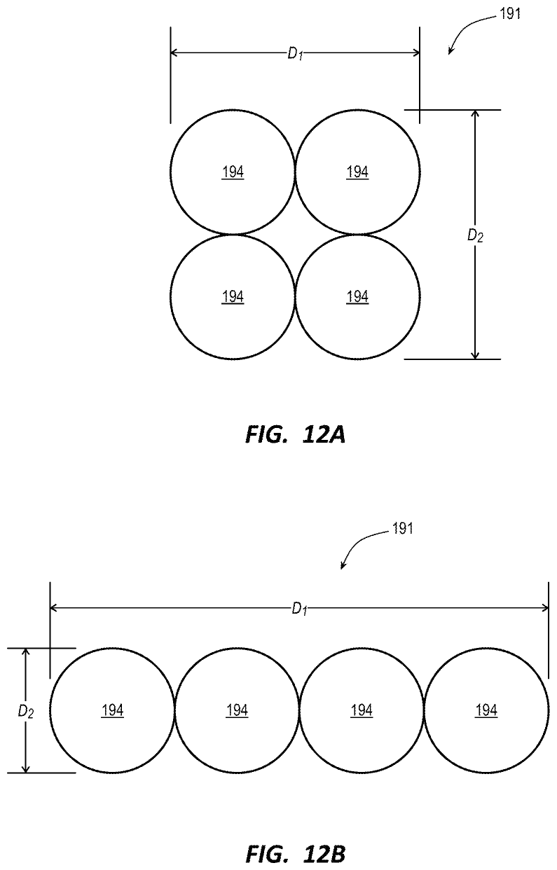

As depicted in FIGS. 12-12B, the braided reinforcement structure 190 includes a first stranded yarn 191 and a second stranded yarn 192. Each of the stranded yarns 191, 192 include a plurality of filaments or singles 194 twisted together. In some embodiments, the singles 194 are formed of an aromatic polyamide material. In another embodiment, the singles 194 are formed of a para-aromatic polyamide material. In some embodiments, the singles 194 may have diameters from about 0.01 mm to about 0.03 mm. When twisted together, the singles 194 form the stranded yarns 191, 192, which, in some embodiments, may have a diameter ranging from about 0.1 millimeters to about 0.4 millimeters.

The stranded yarns 191, 192 can be braided together in an over-under braid pattern as shown in FIG. 12, wherein the first stranded yarn 191 has a left-handed twist and the second stranded yarn 192 has a right-handed twist. The first stranded yarn 191 crosses over the second stranded yarn 192 and the second stranded yarn 192 crosses over the first stranded yarn at cross-over points 195. In other embodiments, the braided reinforcement structure 190 may include more than two stranded yarns. For example, the braided reinforcement structure 190 may include three, four, five, or more stranded yarns. In some embodiments, the braid pattern may be any suitable pattern that increases kink and elongation resistance and reduces ovalization of the shaft section 156. For example, the braid pattern may be an over-under-under pattern, a two over-two under pattern, or any other arrangement.

As illustrated in FIG. 12A, the second stranded yarn 192 includes a first dimension or width D1 and a second dimension or height D2 that may be substantially equivalent to the first dimension D1 at a section of the second stranded yarn 192 extending between cross-over points 195. As illustrated in FIG. 12B, at the cross-over points 195, the first dimension D1 of the second stranded yarn 192 increases and the second dimension D2 decreases. The first dimension D1 is larger than the second dimension D2 as the singles 194 spread out laterally due to a compressive engagement force applied to the second stranded yarn 192 by the first stranded yarn 191 at the cross-over points 195. This configuration provides a cross-sectional profile or thickness of the stacked stranded yarns 191, 192 at the cross-over points 195 that is substantially equivalent to the second dimension D2 that allows the wall 157 to have a constant thickness at the cross-over points 195 and between the cross-over points 195.

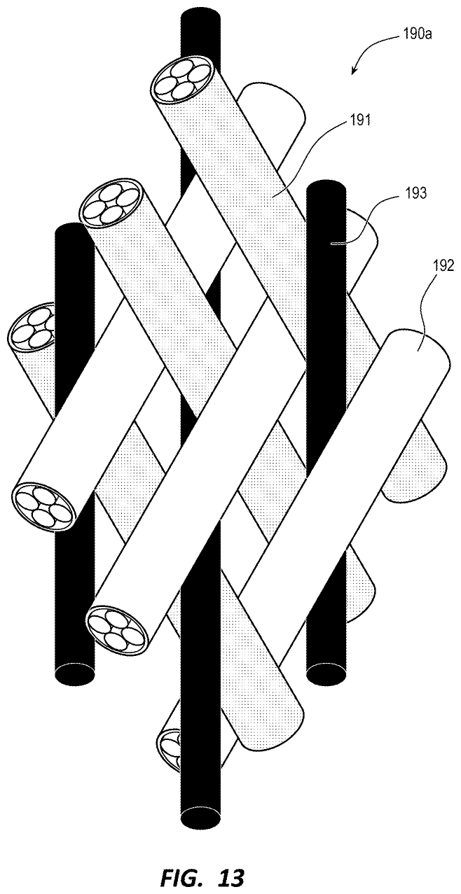

As illustrated in FIG. 13, another embodiment of a braided structure 190a may include a third stranded yarn 193. The third stranded yarn 193 can be axially oriented in parallel with a longitudinal axis of the shaft section 156. In embodiments including an axially orientated third stranded yarn 193, the number of third stranded yarns 193 in the structure can range from one to four. (As detailed throughout, embodiments without an axially oriented third stranded yarn 193 are also within the scope of this disclosure.) Similar to the first and second stranded yarns 191, 192, the third stranded yarn 193 may include a plurality of filaments or singles twisted together. The number of filaments or singles may range from about 3 to about 15. As with the first and second stranded yarns 191, 192, in some embodiments, the singles are formed of an aromatic polyamide material. In another embodiment, the singles are formed of a para-aromatic polyamide material. In some embodiments, the singles may have diameters from about 0.01 mm to about 0.03 mm. When twisted together, the singles form the third stranded yarn 193 which, in some embodiments, may have a diameter ranging from about 0.1 millimeters to about 0.4 millimeters. The third stranded yarn 193 can be woven into the braided structure 190a in any suitable pattern. For example, as illustrated in FIG. 13, the third stranded yarn 193 is woven in an over-under-over pattern. In another embodiment, the third stranded yarn 193 may not be woven into the braided structure 190a but extend longitudinally along the length of the braided structure 190a.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art, and having the benefit of this disclosure, that changes may be made to the details of the above-described embodiments without departing from the underlying principles of the disclosure herein.

1. A catheter, comprising:
a tube, comprising:
a circumferential wall extending along the length of the tube, wherein the wall comprises:
a braided structure embedded within the wall comprising a first stranded yarn and a second stranded yarn, wherein the first and second stranded yarns comprise an aromatic polyamide material; and
a polymer, wherein the polymer surrounds the braided structure.

2. The catheter of claim 1, wherein the aromatic polyamide material comprises a para-aromatic polyamide material.

3. The catheter of claim 1, wherein the stranded yarn comprises a plurality of monofilament singles twisted together.

4. The catheter of claim 3, wherein each of the plurality of monofilament singles comprises the aromatic polyamide material.

5. The catheter of claim 1, further comprising a third stranded yarn embedded within the wall and oriented parallel to a longitudinal axis of the tube.

6. The catheter of claim 5, wherein the number of the third stranded yarn ranges from one to four.

7. The catheter of claim 1, wherein the first stranded yarn is oriented in a first rotational direction and the second stranded yarn is oriented in a second rotational direction, opposite the first rotational direction;
wherein the first stranded yarn crosses over the second stranded yarn at a first cross-over point and the second stranded yarn crosses over the first stranded yarn at a second cross-over point adjacent the first cross-over point; and
wherein a thickness of the first and second stranded yarns at the first and second cross-over points is less than the thickness of the first and second stranded yarns between the first and second cross-over points.

8. The catheter of claim 1, wherein each of the plurality of first cross-over points and each of the second cross-over points comprises:
a first angle defined by the first and second stranded yarns when the catheter is in a relaxed state;
a second angle defined by the first and second stranded yarns when the catheter is in a longitudinally stressed state, wherein the first angle is equivalent to the second angle.

9. The catheter of claim 1,
wherein first and second stranded yarns define an opening, and
wherein the polymer is disposed within the opening.

10. The catheter of claim 9, wherein the number of the openings per linear inch of the tube ranges from one to 50.

11. A medical device delivery catheter, comprising:
a handle;
an outer sheath operably coupled to the handle;
an inner sheath fixedly coupled to the handle and co-axially disposed within the outer sheath,
wherein the handle is configured to axially displace the outer sheath relative to the inner sheath,
wherein the outer sheath comprises:
a tube, comprising:
a circumferential wall extending along the length of the tube, wherein the wall comprises:
a braided structure embedded within the wall comprising a first stranded (piled) yarn and a second stranded yarn, wherein the first and second stranded yarns comprise an aromatic polyamide material; and a polymer, wherein the polymer surrounds the braided structure.

12. The medical device delivery catheter of claim 11,
wherein the first stranded yarn is oriented in a first rotational direction and the second stranded yarn is oriented in a second rotational direction, opposite the first rotational direction;
wherein the first stranded yarn crosses over the second stranded yarn at a first cross-over point and the second stranded yarn crosses over the second stranded yarn at a second cross-over point adjacent the first cross-over point; and
wherein a thickness of the first and second stranded yarns at the first and second cross-over points is less than the thickness of the first and second stranded yarns between the first and second cross-over points.

13. The medical device delivery catheter of claim 11, wherein each of the plurality of first cross-over points and each of the plurality of second cross-over points comprise:
a first angle defined by the first and second stranded yarns when the catheter is in a relaxed state;
a second angle defined by the first and second stranded yarns when the catheter is in a longitudinally stressed state, wherein the first angle is equivalent to the second angle.

14. The medical device delivery catheter of claim 11,
wherein first and second stranded yarns define an opening, and
wherein the polymer is disposed within the opening.

15. The medical device delivery catheter of claim 11, wherein the tube is configured to restrain axial elongation of the outer sheath when axially displaced by the handle.

16. The medical device delivery catheter of claim 11, further comprising a third stranded yarn embedded within the wall and oriented parallel to a longitudinal axis of the tube.
